(19) United States
(12) Patent Application Publication
(10) Pub. No.: US 2012/0071756 A1
(43) Pub. Date: Mar. 22, 2012

(54) APPARATUS AND METHOD FOR DETECTING EXAMINATION TISSUE USING ULTRASOUND SIGNAL

(75) Inventors: Dong Hoon YU, Daejeon (KR); Soo Yeul LEE, Daejeon (KR); Jeong Won LEE, Daejeon (KR); Seung Hwan KIM, Daejeon (KR)

(73) Assignee: Electronics and Telecommunications Research Institute, Daejeon (KR)

(21) Appl. No.: 13/086,220

(22) Filed: Apr. 13, 2011

(30) Foreign Application Priority Data
Sep. 17, 2010 (KR) 10-2010-0091626

(51) Int. Cl. A61B 8/00

(52) U.S. Cl. 600/437

(57) ABSTRACT

An apparatus for detecting an examination tissue using an ultrasound signal is disclosed. The apparatus includes a scanning unit to generate an image related to an object, using the ultrasound signal, a sampling unit to convert a resolution of the generated image, and a controlling unit to retrieve an n-number of figure lines from the resolution-converted image and thereby determine whether each of the retrieved figures line is an examination tissue, wherein n is a natural number.

### CROSS-REFERENCE TO RELATED APPLICATION

This application claims the benefit of Korean Patent Application No. 10-2010-0091626, filed on Sep. 17, 2010, in the Korean Intellectual Property Office, the disclosure of which is incorporated herein by reference.

### BACKGROUND

1. Field of the Invention

The present invention relates to an apparatus and method for automatically detecting an examination tissue, by generating an image related to an object by an ultrasound signal and determining whether a figure line detected from the image corresponds to the examination tissue.

2. Description of the Related Art

Recently, occurrence of breast cancer is gradually increasing. An early diagnosis of the breast cancer is essential since breast cancer can be cured more effectively when detected early.

Nowadays, an X-ray imaging method and an ultrasonic imaging method are mainly used to diagnose the breast cancer. The X-ray imaging method provides a high image resolution, a low noise, and a high efficiency of finding calcification occurring among masses. However, according to the X-ray imaging method, masses and tissues of a portion where most of the masses are present are all shown in white. Therefore, it is hard to recognize accurate positions of the masses. In addition, exposure to radiation is a problem of the X-ray imaging method.

Conversely, an ultrasonic imaging method enables easier detection of masses since a portion where most of the masses are present are all shown in white, while masses are shown in darker colors. Additionally, the ultrasonic imaging method is harmless to a human body.

However, different from an X-ray image enabling diagnosis with a small number of images, the ultrasonic imaging method requires imaging of the overall breast area, thereby increasing a quantity of image data to be inspected by a doctor that diagnoses the masses. Also, detection of the masses is inconvenient since the doctor has to directly recognize the masses.

Accordingly, there is a desire for an apparatus and method capable of automatically detecting examination tissue.

### SUMMARY

An aspect of the present invention is to automatically detect examination tissue such as masses, by generating an image regarding an object using an ultrasonic signal and determining whether a figure line retrieved from the image is the examination tissue.

According to an aspect of the present invention, there is provided an apparatus of detecting an examination tissue using an ultrasound signal, the apparatus including a scanning unit to generate an image related to an object, using the ultrasound signal, a sampling unit to convert a resolution of the generated image, and a controlling unit to retrieve an n-number of figure lines from the resolution-converted image and thereby determine whether each of the retrieved figures line is an examination tissue, wherein n is a natural number.

According to another aspect of the present invention, there is provided a method of detecting an examination tissue using an ultrasound signal, the method including generating an image related to an object, using an ultrasonic signal, converting a resolution of the generated image, and retrieving an n-number of figure lines from the resolution-converted image and thereby determining whether each of the retrieved figure lines is an examination tissue, wherein n is a natural number.

### EFFECT

According to embodiments of the present invention, an image regarding an object is generated by an ultrasound signal and whether a figure line retrieved from the image is an examination tissue is determined. Accordingly, an examination tissue such as a mass may be automatically detected. Since the examination tissue is thus detected and supplied, position and size of the examination tissue may be efficiently recognized.

### BRIEF DESCRIPTION OF THE DRAWINGS

These and/or other aspects, features, and advantages of the invention will become apparent and more readily appreciated from the following description of exemplary embodiments, taken in conjunction with the accompanying drawings of which:

FIG. 1 is a diagram illustrating a structure of an examination tissue detection apparatus according to an embodiment of the present invention;

FIG. 2 illustrates images for describing an exemplary method of detecting an examination tissue by an examination tissue detection apparatus using an ultrasound signal, according to an embodiment of the present invention;

FIG. 3 is a flowchart illustrating a method of detecting an examination tissue using an ultrasound signal, according to an embodiment of the present invention; and

FIG. 4 is a flowchart illustrating a method of determining whether a figure line is an examination tissue in an examination tissue detection method using an ultrasound signal, according to an embodiment of the present invention.

### DETAILED DESCRIPTION

Reference will now be made in detail to exemplary embodiments of the present invention, examples of which are illustrated in the accompanying drawings, wherein like reference numerals refer to the like elements throughout. Exemplary embodiments are described below to explain the present invention by referring to the figures.

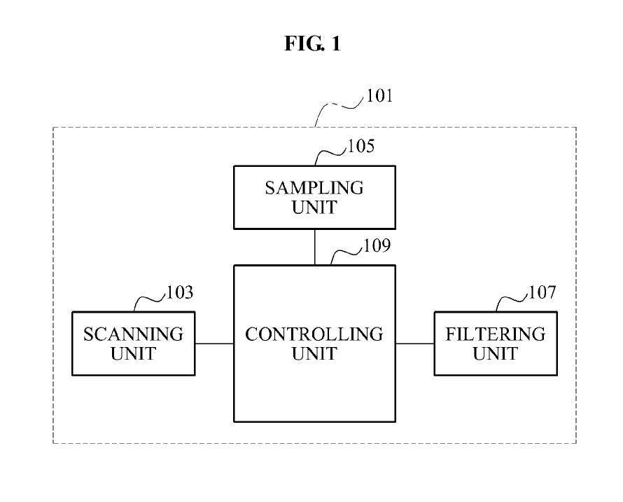

FIG. 1 is a diagram illustrating a structure of an examination tissue detection apparatus 101 using an ultrasound signal, according to an embodiment of the present invention.

Referring to FIG. 1, the examination tissue detection apparatus 101 includes a scanning unit 103, a sampling unit 105, a filtering unit 107, and a controlling unit 109.

The scanning unit 103 may generate an image regarding an object using an ultrasound signal. For example, the object may be a breast of a person.

The sampling unit 105 may convert a resolution of the generated image. That is, the sampling unit 105 may reduce the resolution by down-sampling the image such that an outline of a figure line included in the image becomes sharp.

For example, the sampling unit 105 may reduce a horizontal and vertical size of the image respectively to about ½ through the down-sampling. In addition, the sampling unit 105 may further reduce the horizontal and vertical size of the image to about ¼ by further performing the down-sampling.

The filtering unit 107 may filter the resolution-converted image to remove noise of the image. For example, the filtering unit 107 may perform median filtering, histogram equalization, and Gaussian filtering, in sequence, with respect to the image, thereby removing the noise. When the median filtering is performed, a filter having a size of 7×7 may be used.

The controlling unit 109 retrieves an n-number of figure lines from the noise-removed image, and determines whether each of the retrieved figure lines is an examination tissue. Here, n is a natural number. In addition, the controlling unit 109 may supply the determination result through a display unit (not shown).

For example, the controlling unit 109 may retrieve an oval line or a circular line as the figure line by performing a Hough transform with respect to the image.

More specifically, the controlling unit 109 may estimate whether a first figure line among the n-number of figure lines overlaps a second figure line which is distinguishable from the first figure line. When the first figure line and the second figure line do not overlap each other, the controlling unit 109 may determine the first figure line as the examination tissue, such as a mass, and display the first figure line through the display unit, thus efficiently supplying position and size of the examination tissue.

Here, even through the first figure line does not overlap the second figure line, when a first mean value of internal brightness of the first figure line is equal to or greater than a predetermined value, the controlling unit 109 may determine the first figure line as being different from the examination tissue. In other words, considering that the examination tissue is shown dark, the controlling unit 109 may determine a bright part as being different from the examination tissue. Thus, the examination tissue may be more accurately detected.

When the first figure line and the second figure line overlap each other, the controlling unit 109 may generate an expanded figure line by combining the first figure line and the second figure line or remove one of the first figure line and the second figure line.

More specifically, the controlling unit 109 may calculate a difference between the first mean value and a second mean value regarding internal brightness of the first figure line and the second figure line and, when the difference is less than a predetermined value, may combine the first figure line and the second figure line.

When the difference between the first mean value and the second mean value is equal to or greater than the predetermined value, the controlling unit 109 may select a larger one of the first mean value and the second mean value and remove the figure line corresponding to the selected mean value. When a figure line having a minimum mean value of the internal brightness is selected as the first figure line among the n-number of figure lines, the controlling unit 109 may remove the second figure line, omitting a procedure of comparing the first mean value and the second mean value.

Additionally, when there are an m-number of second figure lines overlapping the first figure line, two figure lines having relatively small mean values among the first figure line and the m-number of second figure lines are selected. Here, m is a natural number greater than 1. When the figure line having the minimum mean value is selected as the first figure line among the n-number of figure lines, the controlling unit 109 may select the two figure lines by selecting the first figure line first and then selecting a figure line having a minimum mean value of the internal brightness among the m-number of second figure lines.

Next, the control unit 109 may calculate the difference between the first mean value and the second mean value of the internal brightness of the two selected figure lines. Based on the calculation result, the controlling unit 109 may combine the two figure lines, or may remove a larger one of the first mean value and the second mean value of the figure lines.

The controlling unit 109 may prepare a figure list regarding the n-number of figure lines, and add or remove a figure line depending on whether each of the figure lines on the figure list is the examination tissue. Whether each of the figure lines is the examination tissue may be determined until no figure line remains on the figure list. Here, the controlling unit 109 may determine the examination tissue from a figure line having a relatively small mean value among the n-number of figure lines on the figure list.

When the first figure line is determined as the examination tissue, the controlling unit 109 may remove the first figure line from the figure list. When the expanded figure is generated by combining the first figure line and the second figure line, the controlling unit 109 may remove the first figure line and the second figure line from the figure list and add the expanded figure. Also, when one of the first figure line and the second figure line is removed, the controlling unit 109 may remove the removed figure line from the figure list.

According to the embodiment, the examination tissue detection apparatus using the ultrasound signal may retrieve the n-number of figure lines from a single image, for example, the image reduced to about ½ in the horizontal and vertical size from the image regarding the object by down-sampling, to thereby determine the examination tissue. However, the present invention is not limited thereto. That is, the examination tissue may be determined by retrieving figure lines from the plurality of down-sampled images, for example the images reduced to about ½ or about ¼ in the horizontal and vertical size. Therefore, accuracy of the determination may increase.

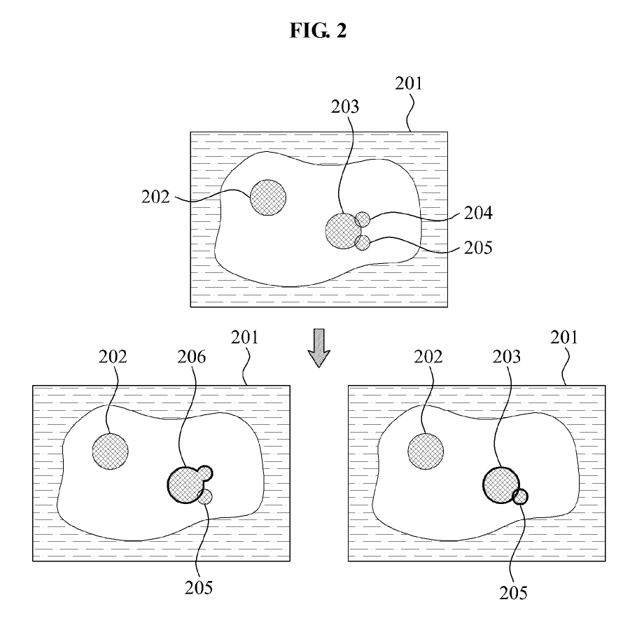

FIG. 2 illustrates images for describing an exemplary method of detecting an examination tissue by an examination tissue detection apparatus using an ultrasound signal, according to an embodiment of the present invention.

Referring to FIG. 2, the examination tissue detection apparatus using the ultrasound signal may retrieve figure lines from a generated image 201 and determine whether each of the retrieved figure lines is an examination tissue.

For example, the examination tissue detection apparatus may retrieve a figure line_#1 202, a figure line_#2 203, a figure line_#3 204, and a figure line_#4 205 from the generated image 201, using the ultrasound signal, and determine whether each of the figure lines is an examination tissue.

To be more specific, the examination tissue detection apparatus may estimate whether the figure line_#1 202 overlaps the other figure lines, that is, the figure line_#2 203, the figure line_#3 204, and the figure line_#4 205. Since the figure line_#1 202 does not overlap the other figure lines as a result of the estimation, the figure line_#1 202 may be determined as the examination tissue.

In addition, the examination tissue detection apparatus estimates whether the figure line_#2 203 overlaps the other figure lines except the figure line_#1 202 determined as the examination tissue, that is, the figure line_#3 204 and the figure line_#4 205. Since the figure line_#2 203 overlaps the other figure lines as a result of the estimation, two figure lines having relatively smaller mean values of internal brightness may be selected from the figure line_#2 203, the figure line_#3 204, and the figure line_#4 205. Next, the examination tissue detection apparatus may calculate a difference between a first mean value and a second mean value regarding the internal brightness of the two figure lines, and then combine the two figure lines or remove a figure line of the two figure lines, the removed figure line corresponding to a larger one of the first mean value and the second mean value, according to the calculation result.

Here, when mean values of internal brightness of the figure line_#2 203, the figure line_#3 204, and the figure line_#4 205 are 0.1, 0.3, and 0.4, respectively, the examination tissue detection apparatus may select the figure line_#2 203 and the figure line_#3 204, having relatively smaller mean values, and calculate the difference of mean value of the internal brightness between the figure line_#2 203 and the figure line_#3 204.

i) In the Case of Combining the Overlapping Figure Lines

When the difference between the mean values is less than a predetermined value as a result of the calculation, the examination tissue detection apparatus may generate an expanded figure line_#1 206 by combining the figure line_#2 203 and the figure line_#3 204.

Next, the examination tissue detection apparatus estimates whether the expanded figure line_#1 206 overlaps the figure line_#4 205. Since the expanded figure line_#1 206 and the figure line_#4 205 are overlapping each other, the examination tissue detection apparatus may combine or remove a figure line of the expanded figure line_#1 206 and the figure line_#4 205, the removed figure line having a larger mean value, using the mean values of the expanded figure line_#1 206 and the figure line_#4 205.

ii) In the Case of Removing One of the Overlapping Figure Lines

When the difference is equal to or greater than the predetermined value, the figure line detection apparatus may remove the figure line_#3 204 having a larger mean value of internal brightness than the figure line_#2 203.

Next, the examination tissue detection apparatus estimates whether the figure line_#2 203 and the figure line_#4 205 overlap. Since the figure lines overlap as a result of the estimation, the examination tissue detection apparatus may combine the figure line_#2 203 and the figure line_#4 205, or remove the figure line_#4 205 having a larger mean value between the figure line_#2 203 and the figure line_#4 205, using the mean values of the expanded figure line_#2 203 and the figure line_#4 205.

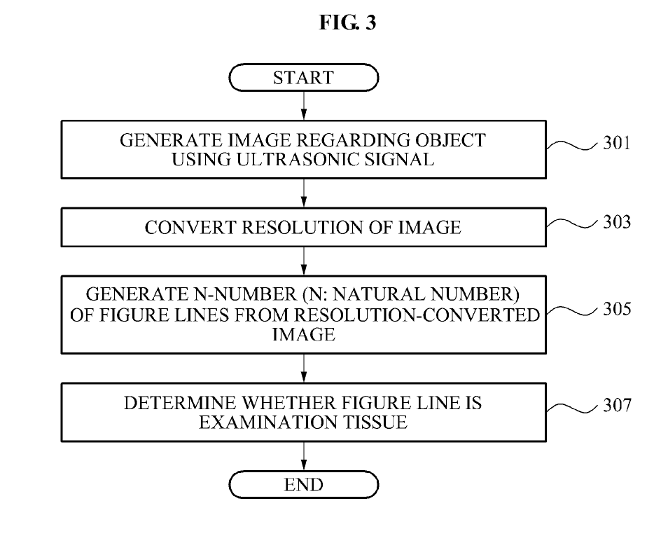

FIG. 3 is a flowchart illustrating a method of detecting an examination tissue using an ultrasound signal, according to an embodiment of the present invention.

Referring to FIG. 3, an examination tissue detection apparatus may generate an image regarding an object, such as a breast of a person, using an ultrasound signal in operation 301.

In operation 303, the examination tissue detection apparatus may convert a resolution of the image.

In other words, the examination tissue detection apparatus may reduce the resolution by down-sampling the image, such that an outline of a figure line included in the image becomes sharp. For example, the examination tissue detection apparatus may reduce horizontal and vertical size of the image to about ½, respectively, by down-sampling the image. Also, the examination tissue apparatus may further reduce the horizontal and vertical size of the image to about ¼ by further performing the down-sampling.

The examination tissue detection apparatus may remove noise of the resolution-converted image by filtering the image. For example, the examination tissue detection apparatus may perform at least one of median filtering, histogram equalization, and Gaussian filtering for the noise removal.

In operation 305, the examination tissue detection apparatus may retrieve an n-number of figure lines from the noise-removed image.

Here, the examination tissue detection apparatus may retrieve an oval line or a circular line as the n-number of figure lines by performing Hough transform with respect to the image.

In operation 307, the examination tissue detection apparatus may determine whether each of the retrieved figure lines is an examination tissue and supply the determination result.

For example, the examination tissue detection apparatus may estimate whether a first figure line among the n-number of figure lines overlaps a second figure line distinguishable from the first figure line. When the first figure line does not overlap the second figure line, the first figure line is determined as the examination tissue and displayed through a display unit. Thus, position and size of the examination tissue may be efficiently supplied.

When the first figure line overlaps the second figure line, the first figure line and the second figure line may be combined into an expanded figure line or one of the first figure line and the second figure line may be removed.

Next, the examination tissue detection apparatus may repeatedly check whether the expanded figure line or a non-removed figure line out of the first figure line and the second figure line is the examination tissue.

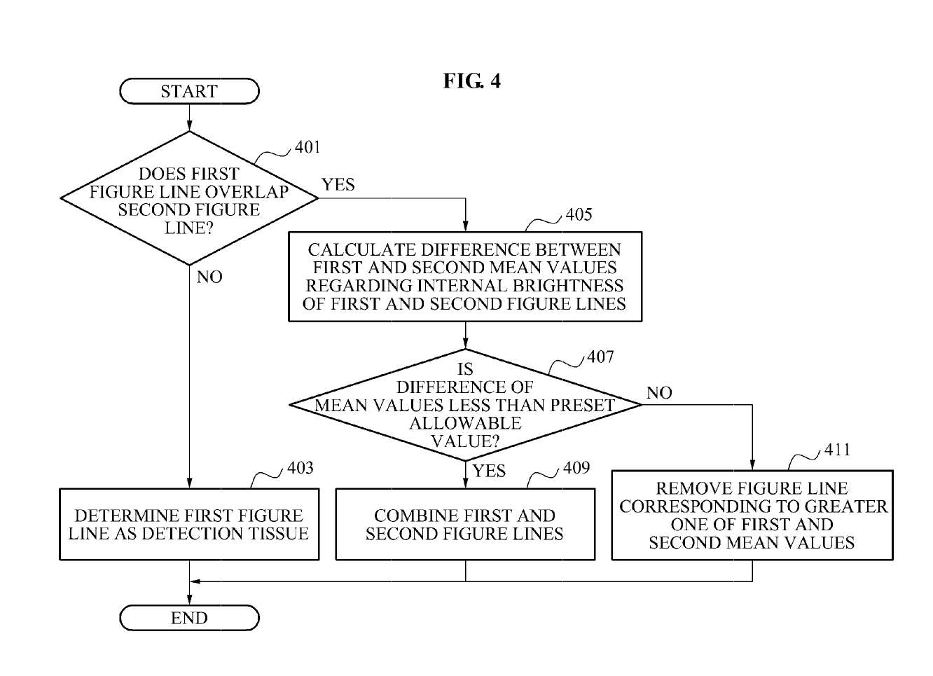

FIG. 4 is a flowchart illustrating a method of determining whether a figure line is an examination tissue in an examination tissue detection method using an ultrasound signal, according to an embodiment of the present invention.

Referring to FIG. 4, in operation 401, the examination tissue detection apparatus may estimate whether a first figure line among an n-number of figure lines retrieved from an image overlaps a second figure line distinguishable from the first figure line. When the first figure line does not overlap the second figure line, the first figure line is determined as the examination tissue in operation 413.

The examination tissue detection apparatus may prepare a figure list regarding the n-number of figure lines and, when the first figure line is determined as the examination tissue, may remove the first figure line from the figure list.

When the first figure line and the second figure line overlap in operation 401, the examination tissue detection apparatus may calculate a first mean value and a second mean value regarding internal brightness of the first figure line and the second figure line and also calculate a difference between the mean values.

When there are an m-number of the second figure lines overlapping the first figure line, the examination tissue detection apparatus may select two figure lines having relatively smaller mean values of the internal brightness among the first figure line and the m-number of second figure lines, thereby calculating a difference between the first mean value and the second mean value of the selected two figure lines. Here, m is natural number greater than 1.

In operation 407, the examination tissue detection apparatus may estimate whether the difference is less than a predetermined value. When the difference is less than the predetermined value, examination tissue detection apparatus may generate an expanded figure line by combining the first figure line and the second figure line in operation 409.

Here, the examination tissue detection apparatus may remove the first figure line and the second figure line from the figure list and add the expanded figure line.

When the difference is greater than the predetermined value in operation 407, the examination tissue detection apparatus may select a larger one of the first mean value and the second mean value, and remove a figure line corresponding to the selected mean value in operation 411.

Here, the examination tissue detection apparatus may remove the figure line removed from the figure list.

The examination tissue detection apparatus, checking the figure list, may repeatedly perform operations 401 through 411 when the figure line is included in the figure list and may finish the repeated performance of operations 401 through 411 when the figure line is absent from the figure list.

According to the embodiments of the present invention, an examination tissue such as a mass may be automatically detected by generating an image regarding an object by an ultrasound signal and determining whether a figure line retrieved from the image is the examination tissue.

The above-described embodiments of the present invention may be recorded in non-transitory computer-readable media including program instructions to implement various operations embodied by a computer. The media may also include, alone or in combination with the program instructions, data files, data structures, and the like. The program instructions recorded on the media may be those specially designed and constructed for the purposes of the embodiments, or they may be of the kind well-known and available to those having skill in the computer software arts. Examples of non-transitory computer-readable media include magnetic media such as hard disks, floppy disks, and magnetic tape; optical media such as CD ROM disks and DVDs; magneto-optical media such as optical disks; and hardware devices that are specially configured to store and perform program instructions, such as read-only memory (ROM), random access memory (RAM), flash memory, and the like. Examples of program instructions include both machine code, such as produced by a compiler, and files containing higher level code that may be executed by the computer using an interpreter. The described hardware devices may be configured to act as one or more software modules in order to perform the operations of the above-described embodiments of the present invention, or vice versa.

Although a few exemplary embodiments of the present invention have been shown and described, the present invention is not limited to the described exemplary embodiments. Instead, it would be appreciated by those skilled in the art that changes may be made to these exemplary embodiments without departing from the principles and spirit of the invention, the scope of which is defined by the claims and their equivalents.

What is claimed is:

1. An apparatus of detecting an examination tissue using an ultrasound signal, the apparatus comprising:
a scanning unit to generate an image related to an object, using the ultrasound signal;
a sampling unit to convert a resolution of the generated image; and
a controlling unit to retrieve an n-number of figure lines from the resolution-converted image and thereby determine whether each of the retrieved figures line is an examination tissue,
wherein n is a natural number.

2. The apparatus of claim 1, wherein the sampling unit reduces the resolution by down-sampling the image.

3. The apparatus of claim 1, wherein the controlling unit estimates whether a first figure line among the n-number of figure lines overlaps a second figure line which is distinguishable from the first figure line,
determines the first figure line as an examination tissue when the first figure line and the second figure line do not overlap, and
supplies the first figure line through a display unit.

4. The apparatus of claim 1, wherein the controlling unit
estimates whether a first figure line among the n-number of figure lines overlaps a second figure line which is distinguishable from the first figure line,
calculates a difference between a first mean value and a second mean value of internal brightness of the first figure line and the second figure line when the first figure line and the second figure line are estimated to overlap, and
combines the first figure line and the second figure line when the calculated difference between the first mean value and the second mean value is less than a predetermined value.

5. The apparatus of claim 4, wherein the controlling unit selects a larger one between the first mean value and the second mean value when the calculated difference between the first mean value and the second mean value is equal to or greater than the predetermined value, and removes a figure line corresponding to the selected mean value.

6. The apparatus of claim 4, wherein, when an m-number of the second figure lines overlap the first figure line, the controlling unit selects two figure lines having relatively small mean values of the internal brightness among the first figure line and the m-number of second figure lines, and calculates a difference between a first mean value and a second mean value of internal brightness of the two figure lines,
and wherein m is a natural number greater than 1.

7. The apparatus of claim 1, wherein the controlling unit retrieves an oval line or a circular line as the n-number of figure lines from the image.

8. A method of detecting an examination tissue using an ultrasound signal, the method comprising:
generating an image related to an object, using an ultrasonic signal;
converting a resolution of the generated image; and
retrieving an n-number of figure lines from the resolution-converted image and thereby determining whether each of the retrieved figure lines is an examination tissue,
wherein n is a natural number.

9. The method of claim 8, wherein the converting of the resolution of the image comprises:
reducing the resolution by down-sampling the image.

10. The method of claim 8, wherein the determining of whether each of the retrieved figure lines is the examination tissue comprises:
estimating whether a first figure line among the n-number of figure lines overlaps a second figure line which is distinguishable from the first figure line, and determines the first figure line as the examination tissue when the first figure line and the second figure line do not overlap.

11. The method of claim 8, further comprising:
calculating a difference between a first mean value and a second mean value of internal brightness of a first figure line and a second figure which is distinguishable from the first figure line when the first figure line among the n-number of figure lines overlaps the second figure line; and
combining the first figure line and the second figure line when the calculated difference between the first mean value and the second mean value is less than a predetermined value.

12. The method of claim 11, further comprising:
selecting a larger one between the first mean value and the second mean value when the calculated difference between the first mean value and the second mean value is equal to or greater than the predetermined value; and
removing a figure line corresponding to the selected mean value.

13. The method of claim 11, wherein, when an m-number of the second figure lines overlap the first figure line, the calculating of the difference comprises:
selecting two figure lines having relatively small mean values of the internal brightness among the first figure line and the m-number of second figure lines; and
calculating a difference between a first mean value and a second mean value of internal brightness of the two figure lines, and
wherein m is a natural number greater than 1.

14. The method of claim 8, wherein the retrieving of the figure line retrieves an oval line or a circular line as the figure line from the image.
